(19) United States
(12) Patent Application Publication
(10) Pub. No.: US 2013/0344062 A1
(43) Pub. Date: Dec. 26, 2013

(54) Identification and Treatment of Aggressive Lung Cancer Tumors

(71) Applicants: Emanuel Petricoin, III, Gainesville, VA (US); Julia Wulfkuhle, Columbia, MD (US); Angela Zupa, Potenza (IT)

(72) Inventors: Emanuel Petricoin, III, Gainesville, VA (US); Julia Wulfkuhle, Columbia, MD (US); Angela Zupa, Potenza (IT)

(21) Appl. No.: 13/841,749

(22) Filed: Mar. 15, 2013

Related U.S. Application Data
(63) Continuation of application No. 13/075,163, filed on Mar. 29, 2011, now abandoned.
(60) Provisional application No. 61/318,563, filed on Mar. 29, 2010.

(51) Int. Cl. G01N 33/68

(52) U.S. Cl. CPC G01N 33/6884
USPC 424/133.1; 506/9

(57) ABSTRACT

This invention relates to the identification and treatment of aggressive lung cancer tumors in patients. More particularly, it provides a method of identifying patients with non-small cell lung carcinoma (NSCLC) who have an aggressive node-negative (N0) tumor and a likelihood of a poor overall survival. The method comprises the step of determining if one or more of certain identified proteins are activated in tumor cells obtained from the patient's tumor, wherein the activation of one or more of the proteins indicates that the patient has an aggressive N0 tumor and is likely to have a poor overall-survival. The invention also provides a method for selecting a treatment for an NSCLC patient with an N0 tumor and a method for treating such patients. It further provides a kit for identifying an NSCLC patient with an aggressive N0 tumor and a likelihood of a poor overall survival and a pharmaceutical composition for treating such patients.

### CROSS-REFERENCE TO RELATED PATENT APPLICATIONS

This application is a continuation application under 35 U.S.C. §§111(a) and 120 of U.S. Patent application Ser. No. 13/075,163, filed Mar. 29, 2011, which claims the benefit of and priority to U.S. Provisional Patent Application No. 61/318,563, filed Mar. 29, 2010, which is incorporated herein by reference in its entirety.

### FIELD OF THE INVENTION

This invention relates to the identification and treatment of aggressive lung cancer tumors in patients. More particularly, it provides methods of identifying non-small cell lung carcinoma (NSCLC) patients with aggressive node-negative (N0) tumors, and it provides therapies for such patients.

### BACKGROUND OF THE INVENTION

Lung cancer is the leading cause of cancer-related mortality in the US and world-wide (1). In 2004, lung cancer caused 20% of all cancer-related deaths in Europe and 29% in the United States (2, 3). Lung tumors are routinely classified in two major histological subtypes: small cell lung carcinoma (SCLC) and non-small cell lung carcinoma (NSCLC), and NSCLC accounts for approximately 85% of all cases of lung cancer. NSCLC is further divided into squamous-cell carcinoma (SCC), adenocarcinoma (AC), and large cell carcinoma (LCC) (4). Adenocarcinoma has become the most prevalent subtype of NSCLC in recent decades (5). Although early stage lung cancer has a higher 5-year survival, the prognosis of stage I lung cancer is highly variable. Postoperative recurrence of stage I non-small cell lung carcinoma (NSCLC) leads to an early mortality rate of approximately 40% (6), and current clinical pathology techniques cannot distinguish stage I patients into long-term (survivors) and short-term survival (fatality) groups.

There have been numerous studies that have used genomic-based approaches to better characterize the molecular underpinnings of NSCLC and develop new taxonomical means to describe the disease (7-10). While there have been some recent attempts to utilize novel discovery-based proteomic approaches for NSCLC cell line studies (11-13) and limited protein signaling analysis of clinical material by us and others (14,15), to date there has yet to be a systematic broad-scale analysis of the functional protein signaling architecture of NSCLC clinical samples and of aggressive early stage disease. A deeper understanding of the ongoing functional protein signaling events within the tumor is of critical importance because protein expression levels largely cannot be predicted by gene transcript expression (16), and protein signaling events mediated principally by phosphorylation-driven post-translational modifications are modulated by ongoing kinase activities that are at the nexus of molecularly targeted inhibitors that now comprise a large portion of the current oncology drug pipeline.

A specific kinase-driven pathway that has well-known significance in lung cancer is the epidermal growth factor receptor (EGFR) family signaling network. Over-expression of EGFR has been observed in 40-80% of the NSCLC, (17-20), which is often associated with aggressive clinical behaviors, such as advanced stage, increased metastatic rate, higher tumor proliferation rate and poor prognosis (19,20). EGFR over-expression in NSCLC provided a rationale to develop EGFR tyrosine kinase inhibitors (TKIs) that block either receptor extracellular domains or the intracellular kinase activity, and a number of these TKIs have been cleared by the FDA for treatment of advanced or metastatic NSCLC (21-24).

The observation that certain subgroups of patients, particularly female patients, nonsmokers, East Asians or patients with lung adenocarcinoma have a higher response rate and clinical benefit with certain targeted therapies, motivated researchers to elucidate the molecular mechanism responsible for this increased response (25-28). Recent findings have revealed a positive relationship between the presence of activating mutations in the EGFR tyrosine kinase domain and clinical response (25-28). These somatic mutations cause constitutive activation of the EGFR tyrosine kinase by destabilizing its auto-inhibited conformation, which is normally maintained in the absence of ligand stimulation (29). However, most of those patients with mutational portraits that predict best response to EGFR TKIs usually relapse and become resistant to further treatment with these inhibitors (31-33). Thus, while most of clinical research on EGFR has been focused on receptor over-expression and gene mutation profiling/status, only very recently have we begun to understand the relationships between EGFR phosphorylation patterns in lung cancer tissue, relationships between EGFR mutations and EGFR phosphorylation levels at defined sites, and an understanding of which EGFR phosphorylation sites are activated in individual patient tumors (14, 34).

### BRIEF DESCRIPTION OF THE FIGURES

FIG. 1. Activated c-erbB3-AKT Signaling Network in Node-Negative NSCLC correlates with poor overall survival. A focused analysis of 5 biochemically linked signaling proteins (ERBB3 Y1289, PRAS40 T246, FOXO1 S256, GSK3 alpha/beta S21/9, and AKT S473) found within the overarching protein activation signature is shown using a signaling cartoon of AKT signaling network. The Figure shows that node negative NSCLC patients with longer term survival have lower levels of phosphorylation of the ErbB-3/EGFR-AKT signaling network. The entire pathway is involved.

### DESCRIPTION OF THE INVENTION

The present invention provides a method of identifying patients with non-small cell lung carcinoma (NSCLC) who have an aggressive node-negative (N0) tumor and a likelihood of a poor overall survival (OS). It also provides a method for selecting a treatment for an NSCLC patient with an N0 tumor and a method for treating such patients. It further provides a kit for identifying an NSCLC patient with an aggressive N0 tumor and a likelihood of a poor OS and a pharmaceutical composition for treating such patients. The term “node-negative” or “N0” broadly means no evidence of tumor metastasis to lymph nodes and is used herein as it is understood and used by physicians involved in the diagnosis and treatment of NSCLC. The term “aggressive” as used herein means tumors from patients with node negative NSCLC who have short overall survival, as defined as less than 30 months from time of diagnosis. The term “overall survival” or “OS” as used herein means time from diagnosis to time of death. The term “poor overall survival” as used herein means having an overall survival of less than the median number of months for typical patients with N0 NSCLC. In the study described in the Examples, the median OS for patients with N0 disease was 31 months with the average OS for the patients with poor survival being 9 months. The singular forms “a,” “an,” and “the” refer to one or more, unless the context clearly indicates otherwise.

As used herein, the term “patient” refers to a human. The invention can also be used with other subjects, such as any mammal with a non-small cell lung carcinoma. Other suitable mammalian subjects include, but are not limited to, laboratory animals, such as a mouse, rat, rabbit, or guinea pig, farm animals, and domestic animals or pets. Non-human primates, such as monkeys, are also included.

The method of identifying an NSCLC patient with an aggressive N0 tumor and a likelihood of a poor overall-survival comprises the step of determining if one or more of the proteins listed in Table 4 are activated in tumor cells obtained from the patient's tumor, wherein the activation of one or more of the proteins indicates that the patient has an aggressive N0 tumor and is likely to have a poor overall-survival. A sample of the tumor is obtained from the patient, and tumor cells are analyzed to determine if one or more of the proteins are activated. In one embodiment of this method, one or more of the proteins are selected from Table 6.

The method may be used with any of the subtypes of NSCLC tumors. In one embodiment, it is applied to adenocarcinomas. In another embodiment, it is applied to squamous-cell carcinomas.

A “sample” is any suitable cell or tissue that can be assayed to determine the activation status of the target proteins. Suitable samples include, e.g., tumor biopsies which are excised from the tissue using any suitable method in the art. In particular, samples of a particular cell type, whether normal or diseased, may be micro-dissected using laser-capture micro-dissection (“LCM”) techniques, as described in U.S. Pat. Nos. 5,843,657, 6,251,516 B1, and 6,969,614 B1, each of which is hereby incorporated by reference in its entirety. LCM allows for isolation of pure populations or subpopulations of the desired cell type, such as a diseased cell population or a normal cell population, or both from the same tissue sample. The cells of interest can be identified, e.g., by morphology, in situ immunohistochemistry, or fluorescent microscopy. By combining microscopy-based cell identification techniques with laser activation of the polymeric substrate to which the tissue sample is applied, very precise extraction of the desired cells is possible.

Activation of one or more of the proteins indicates that the patient has an aggressive N0 tumor and is likely to have a poor overall survival. In one aspect of the invention, activation is determined by measuring the phosphorylation, total amount, or cleavage of the proteins in Table 4. This also provides a map of activated pathways of signaling proteins in the tumor cells. In one embodiment, the proteins are one or more of the proteins in Table 6, and their activation is determined by measuring their phosphorylation.

Such measurement is done by techniques known to those skilled in the art. These include protein microarray analysis, immunohistochemistry, antibody microarray analysis, bead capture, western blotting, enzyme-linked immunosorbent assay (ELISA), suspension bead array, or any semi-quantitative immunoassay based methodology. In particular embodiments, reverse phase protein microarray analysis is used. In more particular embodiments, reverse phase protein microarray analysis is used to detect phosphorylated signaling proteins and/or the total amounts of the signaling proteins regardless of their phosphorylation state.

A protein microarray is an assay format that utilizes a substrate for simultaneously testing multiple samples as well as for testing multiple target proteins in the same assay. Examples of typical microarray substrates include nitrocellulose, derivatized glass slides, and 3-dimensional substrates such as hydrogels. Nitrocellulose-coated glass slides are particularly useful, as a variety of detection methods can be used with this substrate, including chromogenic, fluorometric, and luminescent detection methods.

In one embodiment, the reverse phase protein microarray analysis comprises the steps of: (i) lysing tumor cells obtained by laser capture microdissection; (ii) contacting the lysates with a microarray; and (iii) analyzing the lysates on the microarray. In one aspect of this embodiment, the lysates are analyzed with an immunoassay. In a more particular aspect, they are probed with phosphorylated, cleaved, or total protein antibodies. The antibodies can be polyclonal or monoclonal antibodies. In a particular aspect, the antibodies are one or more of the antibodies listed in Table 2.

In determining the activation of the proteins, the phosphorylation, cleavage, or total amount of one or more of the proteins in Table 4 or the phosphorylation of one or more of the proteins in Table 6 is measured and compared to the activation (phosphorylation, cleavage, or total amount as the case may be) of corresponding reference proteins/lysates. In one embodiment, the activation of the one or more proteins is compared to a series of calibrated standards, wherein the standards contain predetermined amounts of the one or more proteins/phosphoproteins/phosphopeptides such that the value obtained for each patient sample is interpolated to the calibrator in order to generate a calibrated value. In one aspect of this embodiment, the calibrated value is compared to population data or reference standards with known low and high amounts of the target protein such that a determination of high and low levels of the given protein in Tables 4 or 6 can be made. Further, the calibrated value can then be used to develop “cut-point(s)” value for clinical assignment. Such a cut-point can be determined by using common data mining techniques, such as receiver operating characteristics (ROC) analysis of calibrated values derived from the analysis of populations of tumor lysates, wherein the lysates are derived from tumors from patients with known outcome and/or response to therapy. In another aspect of this embodiment, a range of calibrated values is derived from clinical outcome based on population data, wherein the reference standards are correlated to low and high amounts of the target protein such that a determination of high and low levels of a given protein in Tables 4 or 6 can be determined and correlated to clinical outcome. This correlates activation of the proteins in NSCLC patients with N0 tumors to clinical outcomes for those patients.

As used herein, the phrase “one or more of the proteins” means any whole number from one through the total number of proteins listed in Table 4 or Table 6. For example, it could be two or more, three or more, four or more, five or more, etc. It could be 10 or more, 15 or more, 20 or more, 25 or more, etc. and all numbers between.

In one embodiment of the invention, various combinations of proteins are selected from the various signaling groups shown in Table 6. In one aspect of this embodiment, at least one protein is selected from each of at least two of the separate signaling groups shown in Table 6. In another aspect, at least one protein is selected from each of at least three of the separate signaling groups. In a further aspect, at least one protein is selected from each of at least four of the separate signaling groups. In still a further aspect, at least one protein is selected from each of at least five of the separate signaling groups. In yet another aspect, at least one protein is selected from each of the six separate signaling groups. In still another aspect, all of the proteins are selected from one of the signaling groups shown in Table 6.

The invention also includes a method for selecting a treatment for an NSCLC patient with an N0 tumor. The method comprises the step of determining if one or more of the proteins listed in Table 4 or Table 6 are activated in tumor cells obtained from the patient's tumor. The activation of one or more of the proteins indicates that the patient should be treated with aggressive or targeted therapy.

The invention also includes a method for treating an NSCLC patient with a node negative tumor. Node negative NSCLC patients are usually treated only with surgery. Therefore, as used herein, aggressive therapy is any therapy in addition to surgery. In one embodiment, the therapy is the delivery to the patient of a therapeutically effective amount of any therapeutic agent that cures, treats, or ameliorates the disease. For example, the therapeutic agent may be a small molecule compound, an aptamer, a protein, such as an antibody, ligand, enzyme, or cytokine, or a nucleic acid, such as a small interfering RNA (siRNA) molecule or an anti-sense DNA or RNA molecule. In one aspect, the aggressive therapy is chemotherapy. In addition or alternatively, targeted therapy is used. As used herein, “targeted therapy” is the administration of any drug or chemotherapeutic agent that specifically inhibits the enzymatic activation of one or more of the proteins listed in Tables 4 or 6 or eliminates the expression of the protein all together. In one embodiment, the chemotherapeutic agent is selected from the agents listed in Table 7. In another embodiment, the chemotherapeutic agent is selected from the agents listed in Table 8. A therapeutically effective amount of one or more therapeutic agents is administered to the patient. Such dosages are readily determinable by those skilled in the art, given the teachings contained herein. In one embodiment, two different therapeutic agents are administered that target two different proteins in two different pathways. Additional agents that target different proteins in the same or different pathways can also be administered. In one embodiment, the one or more proteins are selected from the proteins listed in Table 6. In one aspect of this embodiment, the chemotherapeutic agent is selected from the agents listed in Table 7. In another aspect of this embodiment, the chemotherapeutic agent is selected from the agents listed in Table 8.

The invention further includes a kit for identifying an NSCLC patient with an aggressive node-negative tumor and a likelihood of a poor overall survival. The kit comprises: (i) one or more reagents for determining the activation level of one or more of the proteins listed in Tables 4 or 6, and (ii) instructions for performing the assay. In one embodiment, the reagents are antibodies, such as polyclonal or monoclonal antibodies. In one aspect of this embodiment, the antibodies are selected from those identified in Table 2. In another embodiment, kit further comprises a container for the reagents.

The invention also includes a pharmaceutical composition for treating an NSCLC patient with an aggressive N0 tumor and the likelihood of poor overall survival. It comprises a therapeutically effective amount of: (i) a therapeutic agent that targets one or more of the proteins listed in Table 4 or Table 6, and (ii) a pharmaceutically acceptable carrier. In one embodiment, the therapeutic agent is a chemotherapeutic agent. In one aspect of this embodiment, it is selected from agents listed in Table 7. In another aspect of this embodiment, it is selected from the agents listed in Table 8. Given the teachings contained herein, one skilled in the art would readily be able to develop compositions suitable for administration to a patient and to determine the dose of the therapeutic agent required.

The following examples illustrate certain aspects of the invention and should not be construed as limiting the scope thereof.

### EXAMPLES

### Introduction

In the present study, we analyzed the activated protein signaling architecture in laser capture microdissected (LCM) NSCLC epithelial cells from individual biopsy specimens using reverse phase protein microarray (RPMA) to interrogate over a hundred key signaling proteins in patients with node negative and node positive disease. Such broad-scale functional protein signaling mapping allowed us to test our hypothesis that while NSCLC maybe characterized by a heterogeneous mutational background at the genomic level, tumors may be defined by distinct signaling activation subgroups at the proteomic level and that signatures of aggressive disease could be found manifested by distinct signaling activation. Consequently, the goals of this study were to utilize a functional signal pathway activation mapping approach to begin to develop a pilot NSCLC signaling taxonomy knowledge base, a deeper understanding of EGFR signaling architecture, and to determine if there were protein signaling network activation events that could be found in early stage N0 disease that correlated with an aggressive phenotype.

### Materials and Methods

### NSCLC Tissue Study Set

The study population consisted of 47 fully informed patients (36 men and 11 women; mean age 66 years; range 43-83), who underwent surgery for NSCLC at Clinica Chirurgica 2, Padova, Italy, between 1993 and 2005 (Table 1). Twenty-seven of the tumors were adenocarcinoma and 20 were squamous carcinoma. According to TNM staging system 24 (51%) were classified T1, 20 (42.5%) T2, and 3 (6.5%) T3; 28 (59.6%) were N0, 14 (29.8%) were N1, 4 (8.5%) were N2, and 1 (2.1%) Nx. The histological grade was assessed according to WHO criteria: 7 (14.9%) tumors were well differentiated, 25 (53.2%) moderately differentiated, 11 (23.4%) poorly differentiated, and in 4 (8.5%) tumors histological grade was not available (table 1). All specimens were snap-frozen in liquid nitrogen within 5 minutes of surgical removal to preserve molecular information. None of the patients underwent preoperative chemotherapy or radiotherapy.

### Laser Capture Microdissection and Reverse Phase Protein Microarray

Highly enriched (greater than 95%) tumor epithelium cell populations were obtained using LCM as described previously (35,36) Approximately 20,000 cells, taken from several tissue sections to ensure tumor coverage and control for cellular heterogeniety, were procured for each patient sample. Pathway activation mapping was performed by reverse phase protein microrray (RPMA) as previously described (14, 37-40). Briefly, LCM procured tumor epithelia were subjected to lysis with 2.5% solution of 2-mercaptoethanol (Sigma, St. Louis, Mo.) in Tissue Protein Extraction Reagent (t-PER™ Pierce)/2×SDS Tris-Glycine 2×SDS buffer (Invitrogen, Carlsbad, Calif.). The lysates were printed on glass-backed nitrocellulose array slides (Schott, Elmsford, N.Y.) using an Aushon 2470 arrayer (Aushon BioSystems, Burlington, Mass.) equipped with 185 μm pins. Each primary NSCLC lysate was printed in triplicate. Arrays were blocked (I-Block, Applied BioSystems, Foster City, Calif.) for 1 h and subsequently probed with 128 phosphorylated, cleaved or total protein antibodies. Detection was performed using a biotinly-tyramide signal amplification strategy and fluorescence-based signal detection using streptavidin-conjugated IRDye680 (LI-COR Biosciences, Lincoln Nebr.). All antibodies were validated for single band specificity as well as for ligand-induction (for phospho-specific antibodies) by Western Blotting prior to use on the arrays as described previously (39). Each array was scanned using a Vidar Revolution 4550 scanner (Vidar Systems Corporation, Herndon Va.). After scanning, spot intensity was analyzed, data were normalized to total protein and a standardized, single data value was generated for each sample on the array by MicroVigene software V2.999 (VigeneTech, North Billerica, Mass.). A full list of all 128 signaling proteins (including total protein/phosphoprotein and cleaved proteins) is shown in Table 2. Protein signaling analytes were chosen for analysis based on their previously described involvement in key aspects of tumorigenesis: growth, survival, autophagy, apoptosis, differentiation, adhesion, motility, and inflammation.

### EGFR Mutational Analysis

Mutation status of EGF receptor kinase domain (exons 18-21) was assessed for each tissue sample. DNA was directly extracted from 10 mm frozen sections by using Maxwell® 16 Tissue DNA Purification Kit (Promega Corporation, Madison, Wis., USA) according to manufacturer's instruction. Amplification of exons 18-21 and fragment sequencing were accomplished using primer sequences previously described (26). PCR was executed with Phusion® High-Fidelity DNA Polymerases (Finnzymes, Espoo, Finland) with an annealing temperature of 58° C. Amplified fragments were purified using MultiScreen PCRₘ₉₆ Plate (Millipore, Billerica, Mass., USA) according to manufacturer's instruction. Purified PCR products were sequenced by using Big Dye® Terminator v3.1 Cycle Sequencing kit (Applied Biosystem, Foster City, Calif., USA) to determine the presence or absence of EGFR mutations. Cycle sequencing reactions were carried out in 96-well format at 25 cycles of 96° C. for 10 s, 50° C. for 5 s, 60° C. for 4 minutes. Sequencing reactions were precipitated using Montage SEQ₉₆ Sequencing Reaction Cleanup Kit (Millipore, Billerica, Mass., USA) heated 3 minutes at 96° C. and analyzed with an ABI PRISM 3100 Genetic Analyzer with the Sequencing Analysis software (Applied Biosystem, Foster City, Calif., USA).

### Statistical Analysis

The continuous variable RPMA data generated were subjected to both unsupervised and supervised hierarchical clustering analysis. Statistical analyses were performed on final RPMA intensity values obtained using SAS v9 software or JMP v5.0 (SAS Institute, Cary, N.C.). Initially, the distribution of variables was checked. If the distribution of variables for the analyzed groups (e.g. primary vs. metastasis or metastatic vs. non-metastatic) was normal, a two-sample t-test was performed. If the variances of two groups were equal, two-sample t-test with a pooled variance procedure was used to compare the means of intensity between two groups. Otherwise, a two-sample t-test without a pooled variance procedure was adopted. For non-normally distributed variables, the Wilcoxon rank sum test was used. All significance levels were set at p≦0.05. Hazard ratios of Cox Proportional Hazard Model were calculated using R version 2.9.2 software (The R Foundation for Statistical Computing). Hazard ratio h of one variable v1 to another variable v2 means the rate of progression to death of v1 is h times that of v2. P value of Chi-square test is testing the null hypothesis of the coefficient of a variable is equal to zero. If the p value for the test is less than 0.05, the null hypothesis is rejected and this variable, such as pathway signature, sex, age, grade, or stage, is significantly related to survival time. Kaplan-Meier (log-rank) survival estimates were used for univariate survival analysis.

### Results

### Protein Pathway Activation Mapping of NSCLC

Unsupervised hierarchical clustering analysis of 128 signaling proteins whose activation/expression levels were determined by RPMA for all 47 patient tumor samples obtained by LCM revealed the presence of five different pathway activation-based subgroups of lung cancer patients. Group A was comprised of only two adenocarcinoma cases and was characterized by activation of growth factor driven signaling, namely activation of ERBB2, ERBB3 and PDGFR, VEGFR and a large number of known linked downstream cytoplasmic signaling networks such as AMPK, PI3K-mTOR, JAK-STAT and ERK signaling modules. Group B was underpinned by AKT network activation and increased relative Cyclin D1 expression. Group C contained tumors that shared ERBB2-3 based signaling, increased ERBB4 along with activation of SMAD signaling, and NOTCH signaling and increased relative expression of autophagy proteins such as Beclin. Group D was dominated by high relative levels of pro-apoptosis proteins such as nearly all cleaved Caspases measured (3,7,9) and cleaved PARP along with elevated expression and/or activation of EGFR receptor family members, namely EGFR, ERBB3 and ERBB4. Other important tyrosine kinase molecules were activated in Group D such as IGFR and RET and ACK. Finally, Group E showed a systemic activation of nearly all signaling networks measured, except for ERBB2- and ERBB3-driven signaling which was comparably lower. These pathway activation mapping results indicate that NSCLC appears to segregate into distinct pathway-driven molecular phenotypes each with a unique molecular signature, and shed further light on the molecular heterogeneity of the disease.

In order to more fully elucidate the nature of the molecular heterogeneity, we explored the extent of differences in the protein signaling architecture between the two major histological types of NSCLC, adenocarcinoma and squamous cell lung cancers. Mean comparison analysis (Table 3) between squamous and adenocarcinoma groups revealed 26 proteins differently expressed between the two types (p<0.05). Interestingly, a large majority of these proteins were more highly activated in squamous compared to the adenocarcinoma cases. Only two proteins, total ERBB3/HER3, and PKC alpha/beta II T638/641 had higher intensity levels in adenocarcinomas compared to squamous carcinomas. The analysis revealed a systemic EGFR-AKT pathway activation in squamous cell carcinomas compared to adenocarcinomas, with increased phosphorylation of EGFR, ERBB3, and a large number of AKT substrates (BAD, FOXO1, FOXO1/O3, PRAS40, 4E-BP1, p27 and GSK3).

Based on these results, we next explored more specifically the signaling aspects of the EGFR and AKT-mTOR networks within squamous and adenocarcinomas, leveraging the multiplexed nature of the RPMA format. Unsupervised hierarchical clustering analysis of total EGFR and 7 independent phosphorylation sites of the receptor (Y1173, Y1148, Y1068, Y992, Y845, Y1045, S1046/1047) whose activation/expression levels were determined by RPMA for all 47 patient tumor samples obtained by LCM revealed that the EGFR signaling architecture clustered into 4 major groups. These groupings were characterized by overt lack of EGFR protein and concomitant lack of EGFR activation at any site (Group 1), or high expression of EGFR protein along with EGFR activation/phosphorylation (Group 2), low or absent total EGFR yet relatively high levels of EGFR activation/phosphorylation at one or more of the 7 sites measured (Group 3), and high levels of EGFR protein and high levels of phosphorylation of EGFR at nearly all of the 7 phosphorylation sites measured (Group 4). This analysis revealed the molecular heterogeneity of EGFR signaling in NSCLC, and that there is a subset of patients with NSCLC that harbor low EGFR yet relatively high levels of receptor activation (Group 3). Distribution of adenocarcinoma and squamous NSCLC was fairly evenly distributed amongst the signaling clusters except for Group 2, where 4/6 of the tumors were squamous cell NSCLC, which indicates that there are no overt EGFR signaling activation architecture differences between NSCLC squamous and adenocarcinomas. Moreover, the signaling subgroups were not underpinned by statistically significant association with nodal status nor EGFR mutational status (data not shown).

Pathway activation mapping of the AKT-mTOR signaling axis, whereby activation of AKT (S473 and T308), mTOR (S2448 and S2481), 4EBP1 (S65 and T70), p70S6K (S371 and T389), and eIF4G (S1108) was measured, revealed 3 overarching subgroups of activation (FIG. 2B). The results showed both AKT and mTOR networks having high relative activation (Group 1), mainly mTOR pathway activation (Group 2), or neither pathway significantly activated (Group 3). Distribution of squamous cell vs adenocarcinoma NSCLC amongst the 3 groupings was fairly even with no statistical association seen (data not shown). These results support those seen for EGFR network activation, namely that NSCLC is comprised of distinct yet heterogeneous subgroups underpinned by pathway activation differences. Generally, AKT activation was seen concomitant with mTOR pathway activation, which is known to lie downstream of AKT.

### Protein Pathway Activation Mapping of Node-Negative NSCLC

While the signaling architecture of pathways that underpin important current molecular targets for NSCLC such as, EGFR, AKT and mTOR, provide evidence of distinct heterogeneity that has implications for therapeutic stratification and response to these therapies, of critical importance in the management of lung cancer is the identification of patients with node-negative disease who have aggressive tumors. Our study set of tumors contained 28 N0 tumors of both adenocarcinoma and squamous carcinoma lineage, and this set provided a unique opportunity to determine if any protein signaling network(s) correlated with overall survival (OS). As shown in Table 4, using a median value OS=31 months as a cutpoint for short versus long term survival for 27 N0 patients where OS data was obtained, RPMA analysis revealed 65 signaling analytes that were significantly more highly activated/phosphorylated in the short term OS N0 cases (N=11, range 2-31 months, median=9 months) versus long term OS (N=16, range 31-120 months, median 90 months). Unsupervised clustering analysis of the data reveals, as expected, near complete segregation of the short term OS from long term OS node negative group with the notable high systemic activation/phosphorylation of many of the signaling proteins seen.

Based on this observation, an optimal cutpoint of 2.8 relative units (RU) based on a protein pathway activation signature was calculated utilizing a combination of normalized and scaled relative intensity values of the 65 analytes shown in Table 4. The optimal cutpoint was determined by ROC analysis which gave a sensitivity of 91% (10/11) and specificity of 88% (14/16) for distinguishing short term (median 9 months) from long term (median 90 months) survival in the 27 N0 patient population where OS was known. The score was determined by first normalizing the relative intensity values of each of the 65 analytes that were elevated patients with N0 and short term survival. Within each analyte, the intensity value of every sample was divided by the highest patient's value within the entire cohort of 65 patients. Normalized intensity values of each analyte were then summed together for every patient. Thus, the final pathway signature score (PSS) did not weigh any significant endpoint as more important than another. Cox Proportional Hazard Model analysis of the data (Table 5) revealed that only the PSS (p=0.0001) and histology for adenocarcinoma (p=0.01) had statistical significance for overall survival compared to other clinical variables measured, including nodal status, sex, grade, site and stage. Since only 4 of the 27 N0 patients harboured an EGFR mutation, statistical correlation with OS was not determined.

Characterization of the underpinning repertoire of the statistically significant activated proteins that comprised the aggressive signature revealed a large number of receptor tyrosine kinases (RTK), EGFR family members, with multiple independent phosphorylation sites on a number of the receptors that were measured (EGFR (Y1173), (Y1148) and (Y845), ERBB3 (Y1289) and (Y1197), VEGFR (Y996) (Y951) and (Y1175), c-KIT Y719). Concomitantly, many other proteins known to be downstream in RTK/growth factor-driven cellular signaling pathways were coordinately activated. In particular, the hierarchical clustering analysis revealed the combined expression of many proteins that belonged to the AKT signaling pathway, including AKT (5473), GSK3αβ (S21/9), PRAS40 (T246), multiple forkhead family members, including FOXO1/3A (T24/32), FOXO3A S253, FOXO1 S256, along with BAD (S155, S136, and S112) and p27 (T187), TSC2 (Y1571) as well as collateral mTOR networks (mTOR (S2448), 4EBP1 (S65 and T70). Other linked networks seen coordinately activated in aggressive N0 tumors at multiple nodes were the AMPK signaling pathway (AMPK α (S485), AMPK β (S108), ACC (S79), LKB1 (S334)), SMAD signaling, pathways regulating motility and adhesion (e.g. FAK (Y576/577), LIMK 1/2 (T508/T505)), and the JAK-STAT pathway (JAK1 (Y1022/1023), STAT3 (S727), and STATS (Y694).

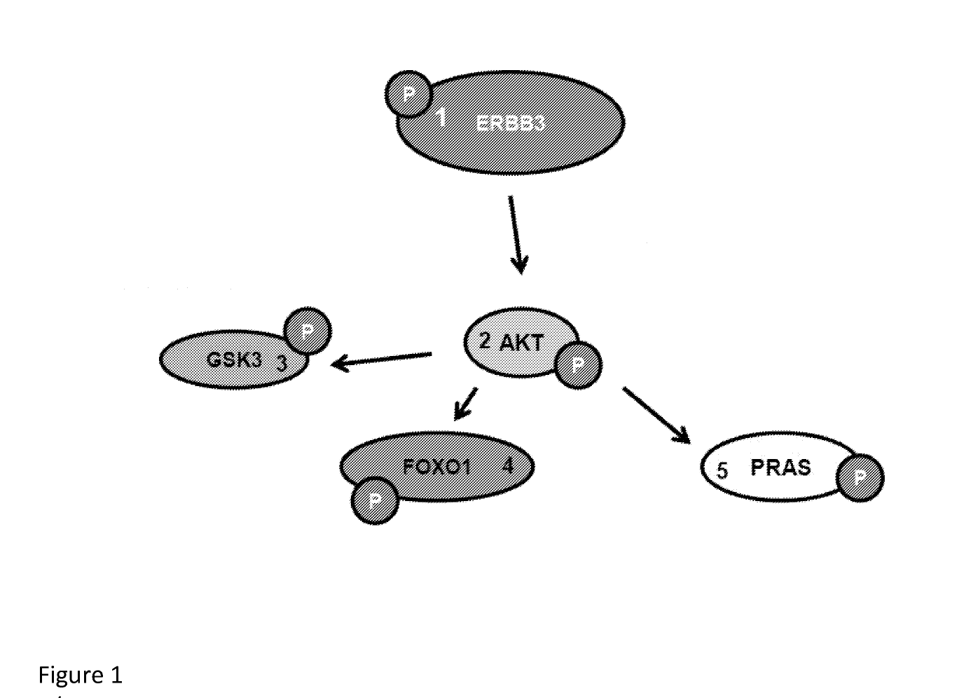

The coordinate nature of the activated kinase-substrate linkage within the signaling architecture of aggressive N0 tumors is visually revealed in FIG. 1, where a selection of the independent statistically significant nodes within the RTK-AKT pathway were mapped to a signaling diagram. For each of the 5 interconected signaling pathway protein “nodes” selected, independent Kaplan-Meier plots were made for N0 patients whose tumors have relative phosphorylation/activation levels above (HIGH) or below (LOW) the median level of the selected analyte across the study set population. The individual statistically significant Kaplan-Meier (KM) plots were ERBB3: p=0.0089; PRAS40: p<0.0001; FOXO1: p=0.0006; GSK3: p=0.0125; and AKT: p=0.089. The data reveal both the nature of the discrimination achieved by the specific individual protein activation as well as the interlinked nature of the data, which indicates a coordinate activation of the pathways in patients with N0 disease and an aggressive intrinsic phenotype.

### Discussion

A broad-scale analysis of the functional protein signaling architecture of NCSLC, quantitatively measuring 128 key signaling proteins concomitantly, revealed the presence of distinct molecular subgroups underpinned by distinct interlinked signaling pathways while at the same time displaying unique patient-specific signaling heterogeneity. The results showed that the overall signaling profiles of squamous and adenocarcinoma tumors appeared to co-mingle while the two were comprised of distinct underpinning signaling motifs. A deeper investigation into EGFR signaling cascades identified unique molecular subgroups of an EGFR-low/pEGFR-high cohort and an EGFR− high/pEGFR-low cohort of NSCLC. Lastly, pathway activation mapping analysis identified biochemically-interlinked RTK-driven protein pathways that could distinguish N0 patients with short term OS (median 9 months) from long term (median 90 months) survival. Analysis of the AKT-mTOR pathway activation network also revealed distinct molecular subgroups of activation with both concomitant linked activation of AKT and mTOR signaling as well as independent activation of either module.

The study utilized a protein microarray driven platform, the reverse phase protein microarray, which has been extensively used by us in other analyses of human malignancies (37-40) to perform the most comprehensive mapping of the protein signaling architecture of human NSCLC clinical specimens to date. The study set utilized a collection of frozen specimens that were carefully chosen based on the control of pre-analytical variables such as sample collection, handling, storage and time-to-freezing after surgical removal. Further, based on past evidence that upfront cellular enrichment is required for accurate protein measurement/activation determination of cellular tissue compartments (38.40), we utilized LCM to greatly enrich for tumor epithelium (>95% purity based on pre and post LCM microscopic visualization) as the cellular input for all analysis.

Our rationale for this study was to select key signaling proteins known to be involved in tumorigenesis and metastasis, regulating growth and energy metabolism, survival, apoptosis, differentiation, motility and inflammation and which were key surrogates and direct targets for the many kinase inhibitors that populate current phase I-III clinical trial pipeline. Furthermore, while signaling can be regulated by a number of post-translational modification driven events (e.g. glycosylation, acetylation, etc), we chose to study these proteins at the functional level by measuring protein phosphorylation: the principal regulator of signal transduction and the key analyte endpoint for the recording of ongoing cellular kinase activity, so that we could generate a direct knowledge snapshot of the ongoing signaling cascades within the tumor cells. Moreover, we postulate that based on the principal “hub” locations within the signaling architecture that many of the proteins we measured are found, even if swaths of the signaling circuitry remain uncharted for this study, we may pick up signaling hits by studying primary feed-in nodes.

Past and recent work involving the analysis of NSCLC using both genomic and proteomic approaches has found evidence for molecular heterogeneity and distinct cohorts of patients underpinned by specific gene expression and protein expression differences (6-13). Recent signaling analysis of NSCLC clinical samples using a much smaller number of phosphoprotein endpoints than what was used in this study found that tumor signaling portraits could be accurately distinguished from matched normal tissue and that energy sensing signaling networks correlated with recurrence (15).

Our analysis indicated that the signaling architecture of NSCLC makes it highly amenable to targeted therapy-based inhibition and consideration of new combinations of therapeutics. We identified distinct cohorts of patients whose tumor portraits contained relatively high levels of activation of receptor tyrosine kinases such as PDGFR, EGFR concomitant with ERBB2 and ERBB3 activation, along with RET and ACK, and downstream pathway activation through cytoplasmic signaling such as AKT-mTOR, RAS-ERK and JAK-STAT activation. While it is not known at this time if the activation levels are driven by activating mutations or exogenous receptor-ligand signaling cascades and tumor-stroma interactions, therapeutic targeting and modulation of the activation could test the causal role the high levels of pathway activation have in each patient tumor.

The identification of cohorts of NSCLC patients with relatively high and low levels of total receptor proteins such as EGFR, yet disconcordant phosphorylation levels of the same protein, would be clinically important. While identification of patients who respond to EGFR inhibitors based on mutational analysis and alternate pathway activation has been a poster child for pharmacogenomics (25-28), recent reports indicate that mutational status may be accurately predicted by EGFR phosphorylation patterns, especially Y1068 and Y1045 (14,41) and Y1173 (41), and thus, protein phosphorylation profiling of EGFR may provide a new companion diagnostic assay method for selection and stratification for therapy (42). Development of quantitative approaches to measure EGFR phosphorylation, such as by RPMA, can be used to identify optimal cut-points for molecular correlates, and a more comprehensive quantitative survey of the many EGFR phosphorylation sites (we measured 7 different sites) along with downstream signaling analysis through AKT and ERK could yield better response prediction to EGFR inhibitors. The fact that AKT and mTOR pathway activation were found in patient subgroups where both modules were activated simultaneously or alone indicates the potential to stratify patients for combination therapeutics that target PI3K-AKT and downstream mTOR together, or PI3K and mTOR inhibitors alone.

Identification of patients with early stage NSCLC who have aggressive tumors that are predestined to a more rapid disease progression would be of critical importance, especially those patients with N0 disease, who are mostly left untreated after surgery. These patients could be stratified to more aggressive paths of treatment and monitoring, and because the signature of aggressive disease we identified is underpinned by interlinked protein kinase activation the findings could point to therapies that might best mitigate the rapid course of disease for these patients and synergize with other recently discovered prognostic markers (43-45). Members of the AMPK-LKB1 pathway, VEGFR, EGFR/ERBB3, PYK2-FAK, AURORA and PLK1, and JAK-STAT, concomitant with many members of the AKT-mTOR downstream signaling modules that integrate these upstream activating events, were all found in this study to be systemically activated in these aggressive tumors. Each of these are under intense investigation for the development of targeted therapy inhibitors, so this signature could form the basis not only for prognostic determination, but clinicians would have an armamentarium of molecularly targeted inhibitors to use for these patients in prospective clinical trials. Some of the molecules we identified in the aggressive signature have also been recently reported as being implicated in aggressive early stage NSCLC and NSCLC patients with worse overall outcome. Analysis of 134 patients with resected stage IA-IIB NSCLC revealed that total levels of mTOR protein as measured by IHC were significantly higher in those patients with node negative or stage IA disease that had poor outcome (46). Analysis by IHC of FoxM1 total protein levels from squamous cell NSCLC revealed a statistical correlation with outcome and an aggressive clinical course (47). Recently, it was reported that NSCLC patients whose tumors had high relative levels of AKT phosphorylation and loss of PTEN expression showed significantly worse 5 year survival rates (48), and that increased levels of phosphorylated eIF4E are associated with survival through AKT pathway activation in NSCLC (49). Interestingly, we found activation/phosphorylation of multiple SMAD family members (SMAD1/5/8 and SMAD2) was elevated in the patients with N0 tumors that had poor survival.

TABLE 1
Patient Characteristics of Tissue Study Set

| Primary Lung cancer samples | Total |
|---|---|
| Number of Patients | |
| Female | 11 |
| Male | 36 |
| Histologic Subtype | |
| Adenocarcinoma | 27 |
| Squamous | 20 |
| T stage | |
| T1 | 24 |
| T2 | 20 |
| T3 | 3 |
| N Stage | |
| N0 | 28 |
| N1 | 14 |
| N2 | 4 |
| NX | 1 |
| Tumor Grade | |
| GX | 4 |
| G1 | 7 |
| G2 | 25 |
| G3 | 11 |
| Months survival | |
| <31 months | 23 |
| >31 months | 24 |
| Status | |
| Died | 38 |
| Alive | 9 |
| Age mean at diagnosis | 66 (48-85) |

TABLE 2
List of antibodies used

| Antibody | Company | Dilution |
|---|---|---|
| 4E-BP1 (S65) | CellSig¹ | 1:50 |
| 4E-BP1 (T70) | CellSig | 1:100 |
| Acetyl-CoA Carboxylase (S79) | CellSig | 1:50 |
| Ack1 (Y284) | CellSig | 1:50 |
| Ack1 (Y857/858) | CellSig | 1:100 |
| Akt (S473) | CellSig | 1:100 |
| Akt (T308) | CellSig | 1:100 |
| ALK (Y1586) | CellSig | 1:500 |
| AMPKalpha1 (S485) | CellSig | 1:50 |
| AMPKBeta1 (S108) | CellSig | 1:50 |
| A-Raf (S299) | CellSig | 1:100 |
| Aurora A (T288)/B (T232)/C (T198) | CellSig | 1:50 |
| Bad (S112) | CellSig | 1:200 |
| Bad (S136) | CellSig | 1:100 |
| Bad (S155) | CellSig | 1:100 |
| Bcl-2 (S70) | CellSig | 1:100 |
| Bcl-2 (T56) | CellSig | 1:200 |
| Total Beclin 1 | CellSig | 1:100 |
| B-Raf (S445) | CellSig | 1:50 |
| c-Abl (T735) | CellSig | 1:50 |
| c-Abl (Y245) | CellSig | 1:100 |
| Cleaved Caspase 9 (D330) | CellSig | 1:100 |
| Total Cleaved Caspase 3 (D175) | CellSig | 1:50 |
| Cleaved Caspase 7 (D198) | CellSig | 1:100 |
| Cleaved Caspase 9 (D315) | CellSig | 1:50 |
| Total c-ErbB2/HER2 | DAKO | 1:1000 |
| Chk1 (S345) | CellSig | 1:50 |
| Chk2 (S33/35) | CellSig | 1:50 |
| cKIT (Y719) | CellSig | 1:50 |
| Cofilin (S3) | CellSig | 1:1000 |
| cPLA2 (S505) | CellSig | 1:1000 |
| CREB (S133) | CellSig | 1:1000 |
| CrkII (Y221) | CellSig | 1:100 |
| Total Cyclin B1 | CellSig | 1:200 |
| Total Cyclin D1 | BD | 1:100 |
| Total Cyclin E | BD | 1:100 |
| EGFR (S1046/1047) | CellSig | 1:500 |
| EGFR (Y1045) | CellSig | 1:50 |
| EGFR (Y1068) | CellSig | 1:50 |
| EGFR (Y1148) | Biosource | 1:100 |
| EGFR (Y1173) | Biosource | 1:100 |
| EGFR (Y845) | CellSig | 1:1000 |
| EGFR (Y992) | CellSig | 1:50 |
| eIF4E (S209) | CellSig | 1:100 |
| eIF4G (S1108) | CellSig | 1:1000 |
| Elk-1 (S383) | CellSig | 1:200 |
| eNOS (S113) | CellSig | 1:50 |
| ErbB2/HER2 (Y1248) | Upstate | 1:500 |
| ErbB3 Y1197 | CST | 1:100 |
| Total ErbB3/HER3 | CellSig | 1:500 |
| ErbB3/HER3 (Y1289) | CellSig | 1:200 |
| Total ErbB4/HER4 | CellSig | 1:50 |
| ERK 1/2 (T202/Y204) | CellSig | 1:1000 |
| Total Estrogen Rec alpha | Dako | 1:50 |
| Estrogen Receptor alpha (S118) | CellSig | 1:1000 |
| FADD (S194) | CellSig | 1:50 |
| FAK (Y397) | BD | 1:500 |
| FAK (Y576/577) | CellSig | 1:200 |
| Foxo3a (S253) | CellSig | 1:50 |
| Foxo1 (S256) | CellSig | 1:100 |
| Foxo1/3a (T24/T32) | CellSig | 1:200 |
| GSK-3alpha/beta (S21/9) | CellSig | 1:200 |
| Histone H3 (S10) | Upstate | 1:500 |
| IGF-1 Rec (Y1131)/Insulin Rec (Y1146) | CellSig | 1:500 |
| IGF-1R (Y1135/36)/IR (Y1150/51) | CellSig | 1:1000 |
| IkappaB-alpha (S32/36) | CellSig | 1:50 |
| IRS-1 (S612) | CellSig | 1:200 |
| Jak1 (Y1022/1023) | CellSig | 1:50 |
| Jak2 (Y1007/1008) | CellSig | 1:100 |
| LIMK1 (T508)/LIMK2 (T505) | CellSig | 1:100 |
| LKB1 (S334) | CellSig | 1:50 |
| MARCKS (S152/156) | CellSig | 1:50 |
| MEK1/2 (S217/221) | CellSig | 1:500 |
| MSK1 (S360) | CellSig | 1:50 |
| Mst1 (T183)/Mst2 (T180) | CellSig | 1:50 |
| mTOR (S2448) | CellSig | 1:100 |
| mTOR (S2481) | CellSig | 1:100 |
| Total Nanog | CellSig | 1:1000 |
| NF-kappaB p65 (S536) | CellSig | 1:50 |
| p27 (T187) | Zymed | 1:200 |
| p38 MAP Kinase (T180/Y182) | CellSig | 1:50 |
| Total p53 | CellSig | 1:5000 |
| p70 S6 Kinase (S371) | CellSig | 1:50 |
| p70 S6 Kinase (T389) | CellSig | 1:50 |
| p90RSK (S380) | CellSig | 1:200 |
| PAK1 (S199/204)/PAK2 (S192/197) | CellSig | 1:50 |
| PAK1 (T423)/PAK2 (T402) | CellSig | 1:100 |
| Cleaved PARP | CellSig | 1:100 |
| Paxillin (Y118) | CellSig | 1:500 |
| PDGF Receptor beta (Y751) | CellSig | 1:50 |
| PDK1 (S241) | CellSig | 1:200 |
| PKA C (T197) | CellSig | 1:200 |
| PKC alpha (S657) | Upstate | 1:1000 |
| PKC alpha/beta II (T638/641) | CellSig | 1:50 |
| PKC theta (T538) | CellSig | 1:100 |
| PKC zeta/lambda (T410/403) | CellSig | 1:50 |
| PKCdelta (T505) | CellSig | 1:100 |
| PLCgamma1 (Y783) | CellSig | 1:100 |
| PLK1 (T210) | BD | 1:200 |
| PRAS40 (T246) | Biosource | 1:1000 |
| PTEN (S380) | CellSig | 1:500 |
| Pyk2 (Y402) | CellSig | 1:200 |
| Raf (S259) | CellSig | 1:100 |
| Ras-GRF1 (S916) | CellSig | 1:50 |
| Ret (Y905) | CellSig | 1:100 |
| RSK3 (T356/S360) | CellSig | 1:500 |
| S6 Ribosomal Protein (S240/244) | CellSig | 1:1000 |
| Shc (Y317) | Upstate | 1:200 |
| Smad1 (S463/S465)/Smad5 | CellSig | 1:50 |
| (S463/S465)/Smad8 (S426/S428) | | |
| Smad2 (S245/250/255) | CellSig | 1:100 |
| Smad2 (S465/467) | CellSig | 1:250 |
| Src (Y527) | CellSig | 1:250 |
| Src Family (Y416) | CellSig | 1:250 |
| Total ST6GALNAC5 | Aviva System | 1:50 |
| Stat1 (Y701) | CellSig | 1:1000 |
| Stat3 (S727) | CellSig | 1:100 |
| Stat3 (Y705) | Upstate | 1:200 |
| Stat5 (Y694) | CellSig | 1:100 |
| Total EGFR | CellSig | 1:100 |
| Total Notch1 | Millipore | 1:10000 |
| Total PTEN | CellSig | 1:50 |
| Tuberin/TSC2 (Y1571) | CellSig | 1:50 |
| VEGFR 2 (Y1175) | CellSig | 1:50 |
| VEGFR 2 (Y951) | CellSig | 1:200 |
| VEGFR 2 (Y996) | CellSig | 1:50 |

¹CellSig = Cell Signaling Technology, Inc.

TABLE 3
Statistically Significant Analytes
Squamous (S) vs Adenocarcinoma NCLC

| | | Expression/ |
|---|---|---|
| Proteins/phosphoproteins | p. value | activation trend |
| PKCalpha/betaII (T638/641) | 0.0296 | S▾ |
| ERBB3/HER3 | 0.0002 | S▾ |
| FOXO1 (T24)/FOXO3a (T32) | 0.0003 | S▴ |
| LKB1 S334 | 0.0023 | S▴ |
| 4E-BP1 T70 | 0.0042 | S▴ |
| PRAS40 T246 | 0.0052 | S▴ |
| p27 T187 | 0.0058 | S▴ |
| Akt S473 | 0.0062 | S▴ |
| PKC zeta/lamda (T410/403) | 0.0091 | S▴ |
| ACK1 Y284 | 0.0093 | S▴ |
| Src Family Y416 | 0.0112 | S▴ |
| MST1 (T183)/MST2 (T180) | 0.0131 | S▴ |
| SMAD1(S/S)/SMAD5(S/S)/SMAD8(S/S) | 0.0174 | S▴ |
| Total EGFR | 0.0174 | S▴ |
| AURORA A (T288)/B (T232)/C (T198) | 0.0174 | S▴ |
| GSK3 alpha-beta S21/9 | 0.0235 | S▴ |
| BAD S136 | 0.0002 | S▴ |
| PKC theta T538 | 0.0412 | S▴ |
| FOXO1 S256 | 0.0491 | S▴ |

TABLE 4
Statistically Significant Analytes for
N0 NSCLC with Short Term Survival

| proteins | N0 p. value |
|---|---|
| 4E-BP1 S65 | 0.0394 |
| 4E-BP1 T70 | 0.0462 |
| Acetyl CoA Carboxylase S79 | 0.0256 |
| Adducin S662 | 0.0049 |
| Akt S473 | 0.0849 |
| AMPK alpha1 S485 | 0.0041 |
| AMPK beta1 S108 | 0.0286 |
| Aurora A (T288)/B (T232)/C (T198) | 0.0107 |
| Bad S112 | 0.0057 |
| Bad S136 | 0.0035 |
| Bad S155 | 0.0228 |
| B-Raf S445 | 0.0273 |
| c-Abl T735 | 0.0049 |
| c-Abl Y245 | 0.0343 |
| CC9 D330 | 0.0122 |
| Chk1 S345 | 0.0273 |
| c-KIT Y719 | 0.0128 |
| cPLA2 (S505) | 0.031 |
| CREB S133 | 0.0162 |
| EGFR Y1148 | 0.0497 |
| EGFR Y1173 | 0.0097 |
| eIF4E S209 | 0.0452 |
| eIF4G S1108 | 0.0179 |
| ErbB3 Y1197 | 0.0273 |
| ErbB3 Y1289 | 0.035 |
| FADD S194 | 0.0057 |
| FAK Y576/577 | 0.0185 |
| FoxO1/3a T24/32 | 0.0029 |
| FoxO3A S253 | 0.0047 |
| FoxO1 S256 | 0.0003 |
| GSK3 alpha-beta S21/9 | 0.0041 |
| Jak1 Y1022/1023 | 0.0538 |
| LIMK1 T508/LIMK2 T505 | 0.0161 |
| LKB1 S334 | 0.0241 |
| MARCKS S152/156 | 0.0011 |
| MEK1/2 S217/221 | 0.014 |
| MSK1 S360 | 0.0043 |
| mTOR S2448 | 0.0106 |
| p27 T187 | 0.0101 |
| p38 MAPKinase (T180/Y182) | 0.0078 |
| PAK1(S199/204)/PAK2(S192/197) | 0.0426 |
| PAK1T423/PAK2T402 | 0.0122 |
| PKC theta T538 | 0.0011 |
| PKC zeta/Lamda (T410/403) | 0.0049 |
| PLCgamma1 Y783 | 0.0114 |
| PLK1 T210 | 0.0248 |
| PRAS40 T246 | 0.0001 |
| Pyk2 Y402 | 0.0014 |
| Raf S259 | 0.0139 |
| Ras-GRF1 (S916) | 0.031 |
| Smad1(S/S)/Smad5(S/S)/Smad8(S/S) | 0.0168 |
| Smad2 S245/250/255 | 0.0037 |
| Smad2 S465/467 | 0.0443 |
| Src Family Y416 | 0.0014 |
| Src Y527 | 0.0162 |
| ST6GALNAC5 | 0.0885 |
| Stat3 S727 | 0.0055 |
| Stat5 Y694 | 0.0185 |
| Tuberin/TSC2 Y1571 | 0.0088 |
| VEGFR Y996 | 0.0016 |
| VEGFR2 Y1175 (19A10) | 0.0018 |
| VEGFR2 Y951 | 0.0001 |

TABLE 5
Cox Proportional Analysis

| | | | Median | p-value |
|---|---|---|---|---|
| | | | survival | log-rank |
| Characteristic | Cases | Deaths | (months) | test |
| Sex | | | | |
| Male | 20 | 15 | 42 | |
| Female | 7 | 5 | 42 | 0.99 |
| Age (years) | | | | |
| <70 | 16 | 12 | 42 | |
| ≧70 | 11 | 8 | 33 | 0.56 |
| Pathway | | | | |
| Activation | | | | |
| Signature | | | | |
| 0 | 11 | 11 | 9 | |
| 1 | 16 | 9 | 90 | 0.0001 |
| T | | | | |
| 1 | 18 | 13 | 46 | |
| 2-3 | 9 | 7 | 9 | 0.27 |
| Grade | | | | |
| 1 | 5 | 4 | 42 | |
| 2 | 15 | 10 | 54 | |
| 3 | 7 | 6 | 15 | 0.18 |
| Histology | | | | |
| Squamo | 9 | 8 | 15 | |
| Adeno | 18 | 12 | 63 | 0.01 |

TABLE 6
Activated Protein Pathways in N0
NSCLC Patients with Short Term OS

| proteins | N0 p. value |
|---|---|
| PKC SIGNALING | |
| MARCKS S152/156 | 0.0011 |
| PKC theta T538 | 0.0011 |
| PKC zeta/Lamda (T410/403) | 0.0049 |
| TGF-BETA SIGNALING | |
| Smad1(S/S)/Smad5(S/S)/Smad8(S/S) | 0.0168 |
| Smad2 S245/250/255 | 0.0037 |
| Smad2 S465/467 | 0.0443 |
| VEGFR SIGNALING | |
| VEGFR Y996 | 0.0016 |
| VEGFR2 Y1175 | 0.0018 |
| VEGFR2 Y951 | 0.0001 |
| AMPK SIGNALING | |
| Acetyl CoA Carboxylase S79 | 0.0256 |
| AMPK alpha1 S485 | 0.0041 |
| AMPK beta1 S108 | 0.0286 |
| LKB1 S334 | 0.0241 |
| PLK1 SIGNALING | |
| Aurora A (T288)/B (T232)/C (T198) | 0.0107 |
| LIMK1 T508/LIMK2 T505 | 0.0161 |
| Chk1 S345 | 0.0273 |
| PLK1 T210 | 0.0248 |
| ERBB3 SGINALING | |
| ErbB3 Y1197 | 0.0273 |
| ErbB3 Y1289 | 0.035 |
| Src Family Y416 | 0.0014 |
| Src Y527 | 0.0162 |
| Stat3 S727 | 0.0055 |
| Stat5 Y694 | 0.0185 |
| cPLA2 (S505) | 0.031 |
| c-Abl T735 | 0.0049 |
| c-Abl Y245 | 0.0343 |

TABLE 7
Chemotherapeutic Agents and Their Targets

| Target | Agent |
|---|---|
| EGFR | Cetuximab (Erbitux ®) |
| | Erlotinib (Tarceva ®) |
| | Gefitinib (Iressa ®) |
| | Matuzumab ( |
| | Panitumumab (Vectibix ®) |
| | Lapatinib (Tykerb ®) |
| HER-2 | Pertuzumab (Omnitarg ®) |
| | Trastuzumab (Herceptin ®) |
| | Lapatinib (Tykerb ®) |
| mTOR | Everolimus (Afinitor ®) |
| | Temsirolimus (Torisel ®) |
| c-Kit | Imatinib mesylate (Gleevec ®) |
| | Sorafenib (Nexavar ®) |
| | Dasatinib (Sprycel ®) |
| | Sunitinib (Sutent ®) |
| | Nilotinib (Tasigna ®) |
| | Pazopanib (Votrient ®) |
| c-abl | Imatinib mesylate (Gleevec ®) |
| | Dasatinib (Sprycel ®) |
| | Nilotinib (Tasigna ®) |
| Raf | Sorafenib (Nexavar ®) |
| VEGFR | Bevacizumab (Avastin ®) |
| | Pazopanib (Votrient ®) |
| | Sunitinib (Sutent ®) |
| | Sorafenib (Nexavar ®) |
| Src | Dasatinib (Sprycel ®) |

TABLE 8
Experimental Chemotherapeutic Agents and their Targets

| Target | Agent | Reference |
|---|---|---|
| Acetyl-CoA | Compounds disclosed in | http://www.wipo.int/pctdb/en/wo.jsp?WO=2003072197 |
| Carboxylase | WO 2003/072197, pub. | |
| | Sep. 4, 2003 | |
| | benzofuranyl α-pyrone | Sugimoto et al., Arch Biochem Biophys. 2007 Dec. 1; |
| | (TEI-B00422) | 468(1): 44-8. |
| AMPK | Compound C | http://www.emdchemicals.com/life-science- |
| | | research/ampk-inhibitor-compound- |
| | | c/EMD_BIO-171260/p_INqb.s1Ls8sAAAEWxmEfVhTm |
| | Dorsomorphin | http://www.tocris.com/pharmacologicalBrowser |
| | dihydrochloride | .php?ItemId=226831 |
| Aurora A | MLN8237 | http://clinicaltrials.gov/ct2/show/NCT01316692 |
| | | ?term=Aurora+A&rank=1 |
| Chk1 | LY2603618 | http://clinicaltrials.gov/ct2/show/NCT01139775 |
| | | ?term=CHK1&rank=3 |
| erbB3 | AV-203 | http://www.aveopharma.com/product_candidates/av-203 |
| | MM-121 | http://www.merrimackpharma.com/pipeline/mm121.html |
| PKC theta | 2,4-diamino-5- | Cywin et al., Bioorg Med Chem Lett. 2007 Jan. 1; |
| | nitropyrimidines | 17(1): 225-30. |
| | Rotterlin | Solomou et al., J. Immunol. 2001 May 1; |
| | | 166(9): 5665-74. |
| PKC zeta | Ruboxistaurin | http://clinicaltrials.gov/ct2/show/NCT00604383 |
| PLK1 | BI 2536 | http://clinicaltrials.gov/ct2/show/NCT00710710?term= |
| | | PLK1&rank=5 |
| | [GSK461364], a Polo-like | http://clinicaltrials.gov/ct2/show/NCT00536835?term= |
| | Kinase 1 (PLK1) Inhibitor | PLK1&rank=1 |
| SMAD/ | P144 TGF-β1-inhibitor | http://clinicaltrials.gov/ct2/show/NCT00656825?term= |
| TGFβ | | SMAD+inhibitor&rank=1 |
| STAT3 | OPB-31121 | http://clinicaltrials.gov/ct2/show/NCT00955812?term= |
| | | STAT3&rank=2 |
| STAT5 | Pimozide | Nelson et al., Blood. 2011 Mar. 24; 117(12): 3421-9. |

### REFERENCES

1. Parkin D M. Global cancer statistics in the year 2000. Lancet Oncol. 2001; 2(9): 533-43.
2. Boyle P, and Ferlay J. (2005) Cancer incidence and mortality in Europe, x Ann. Oncol. 2004; 16, 481-488.
3. Jemal A, Murray T, Ward E, Samuels A, Tiwari R. C; Ghafoor A et al. (2005) Cancer Statistics CA Cancer J. Clin. 2005; 55, 10-30
4. Brambilla E, Travis W D, Colby T V, Corrin B, Shimosato Y. The new World Health Organization classification of lung tumors. Eur Respir J. 2001; 18(6): 1059-68.
5. Franceschi S, Bidoli E. The epidemiology of lung cancer. Ann Oncol. 1999; 10 (Suppl 5): S3-6.
6. Goldstraw P, Crowley J, Chansky K, Giroux D J, Groome P A, Rami-Porta R, et al. International Association for the Study of Lung Cancer International Staging Committee; Participating Institutions. The IASLC Lung Cancer Staging Project: proposals for the revision of the TNM stage groupings in the forthcoming (seventh) edition of the TNM Classification of malignant tumours. J Thorac Oncol. 2007; 2:706-14.
7. Hayes D N, Monti S, Parmigiani G, Gilks C B, Naoki K, Bhattacharjee A, et al. Gene expression profiling reveals reproducible human lung adenocarcinoma subtypes in multiple independent patient cohorts. J Clin Oncol. 2006; 24(31):5079-90.
8. Weir B A, Woo M S, Getz G, Perner S, Ding L, Beroukhim R, et al Characterizing the cancer genome in lung adenocarcinoma. Nature. 2007; 450(7171):893-8.
9. Ding L, Getz G, Wheeler D A, Mardis E R, McLellan M D, Cibulskis K, et al. Somatic mutations affect key pathways in lung adenocarcinoma. Nature. 2008; 455(7216):1069-75.
10. Bryant C M, Albertus D L, Kim S, Chen G, Brambilla C, Guedj M, et al Clinically relevant characterization of lung adenocarcinoma subtypes based on cellular pathways: an international validation study. PLoS One. 2010; 5(7):e11712.
11. Machida K, Eschrich S, Li J, Bai Y, Koomen J, Mayer B J, Haura E B. Characterizing tyrosine phosphorylation signaling in lung cancer using SH2 profiling. PLoS One. 2010; 19; 5(10):e13470.
12. Yu G, Xiao C L, Lu C H, Jia H T, Ge F, Wang W, et al. Phosphoproteome profile of human lung cancer cell line A549. Mol Biosyst. 2011; 7(2):472-9.
13. Wang Y T, Tsai C F, Hong T C, Tsou C C, Lin P Y, Pan S H, et al. An informatics-assisted label-free quantitation strategy that depicts phosphoproteomic profiles in lung cancer cell invasion. Proteome Res. 2010; 9(11):5582-97.
14. VanMeter A J, Rodriguez A S, Bowman E D, Jen J, Harris C C, Deng J, et al. Laser capture microdissection and protein microarray analysis of human non-small cell lung cancer: differential epidermal growth factor receptor (EGPR) phosphorylation events associated with mutated EGFR compared with wild type. Mol Cell Proteomics. 2008; (10):1902-24.
15. Nanjundan M, Byers L A, Carey M S, Siwak D R, Raso M G, Diao L, et al. Proteomic profiling identifies pathways dysregulated in non-small cell lung cancer and an inverse association of AMPK and adhesion pathways with recurrence. J Thorac Oncol. 2010; 5(12):1894-904.
16. Shankavaram U T, Reinhold W C, Nishizuka S, Major S, Morita D, Chary K K, et al. Transcript and protein expression profiles of the NCI-60 cancer cell panel: an integromic microarray study. Mol Cancer Ther. 2007; 6(3):820-32.
17. Piyathilake C J, Frost A R, Manne U, Weiss H, Bell W C, Heimburger D C, et al. Differential expression of growth factors in squamous cell carcinoma and precancerous lesions of the lung. Clin. Cancer Res. 2002; 8:734-44.
18. Salomon D S, Brandt R, Ciardiello F, Normanno N. Epidermal growth factor-related peptides and their receptors in human malignancies. Crit Rev Oncol Hematol 1995; 19:183-232.
19. Ohasaki Y, Tanno S, Fujita Y, Toyoshima E, Fujiuchi S, Nishigaki Y, et al. Epidermal growth factor receptor expression correlates with poor prognosis in non small cell lung cancer patients with p53 overexpression. Oncol Rep 2000; 7:603-7.
20. Nicholson R I, Gee J M, Harper M E. EGFR and cancer prognosis. Eur J Cancer 2001; 37 (Suppl 4):9-15S.
21. Mendelsohn J, Balsega J. The EGFR receptor family as targets for cancer therapy. Oncogene 2000; 19:6550-56.
22. Rosell R, Viteri S, Molina M A, Benlloch S, Taron M. Epidermal growth factor receptor tyrosine kinase inhibitors as first-line treatment in advanced nonsmall-cell lung cancer. Curr Opin Oncol. 2010; (2):112-20.
23. Mendelsohn J. The epidermal growth factor receptor as a target for cancer therapy. Endocr Relat Cancer 2001; 8:3-9
24. Cohen M H, Williams G A, Sridhara R, Chen G, Pazdur R. FDA drug approval summary: gefitinib (ZD1839) (Iressa) tablets. Oncologist 2003; 8:303-6.
25. Paez J G, Janne P A, Lee J C, et al. EGFR mutations in lung cancer: correlation with clinical response to gefitinib therapy. Science 2004; 304:1497-1500.
26. Lynch T J, Bell D W, Sordella R, Gurubhagavatula S, Okimoto R A, Brannigan B W, et al. Activating mutations in the epidermal growth factor receptor underlying responsiveness of non-small-cell lung cancer to gefitinib. N Engl J Med 2004; 350:2129-2139.
27. Mitsudomi T, Kosaka T, Endoh H, Horio Y, Hida T, Mori S et al. Mutations of the epidermal growth factor receptor gene predict prolonged survival after gefitinib treatment in patients with non-small-cell lung cancer with postoperative recurrence. J Clin Oncol 2005, 23:2513-2520.
28. Pao W, Miller V, Zakowski M, Doherty J, Politi K, Sarkaria I, et al. EGFR receptor gene mutations are common in lung cancers from never smokers and are associated with sensitivity of tumors to gefitinib and erlotinib. Proc Natl Acad Sci USA, 2004; 101(36):13306-13311.
29. Yun C H, Boggon T J, Li Y, Woo M S, Greulich H, Meyerson M, et al. Structures of lung cancer-derived EGFR mutants and inhibitor complexes: mechanism of activation and insights into differential inhibitor sensitivity. Cancer Cell 2007; 11:217-27.
30. Carey K D, Garton A J, Romero M S, Kahler J, Thomson S, Ross S, et al. Kinetic analysis of epidermal grow factor receptor somatic mutant proteins shows increased sensitivity to the epidermal growth factor receptor tyrosine kinase inhibitor, erlotinib. Cancer Res 2006; 66:8163-71.
31. Pao W, Miller V A, Politi K A, Riely G J, Somwar R, Zakowski M F, et al. Acquired resistance of lung adenocarcinomas to gefitinib or erlotinib is associated with a second mutation EGFR kinase domain. PLoS Med 2005; 2: e73.
32. Engelman J A, Zejnullahu K, Mitsudomi T, Song Y, Hyland C, Park J O, et al. MET amplification leads to gefitinib resistance in lung cancer by activating ERBB3 signaling. Science. 2007; 316(5827):1039-43.
33. Dahabreh I J, Linardou H, Siannis F, Kosmidis P, Bafaloukos D, Murray S. Somatic EGFR mutation and gene copy gain as predictive biomarkers for response to tyrosine kinase inhibitors in non-small cell lung cancer. Clin Cancer Res. 2010; 16(1):291-303.
34. Zhang G, Fang B, Liu R Z, Lin H, Kinose F, Bai Y, et al. Mass spectrometry mapping of epidermal growth factor receptor phosphorylation related to oncogenic mutations and tyrosine kinase inhibitor sensitivity. J Proteome Res. 2011; 10(1):305-19.
35. Emmert-Buck M R, Bonner R F., Smith P D., Chuaqui R F., Zhuang Z, Goldstein S R, et al. Laser capture microdissection. Science. 1996; 274: 998-1001.
36. Espina V, Wulfkuhle J, Calvert V D, VanMeter A, Zhou W. Coukos G, et al. Laser capture microdissection. Nat. Protoc. 2006; 1, 586-603.
37. Paweletz C P, Charboneau L, Roth M J, Bichsel V E, Simone N L, Chen T, et al. Reverse phase proteomic microarrays which capture disease progression show activation of pro-survival pathways at the cancer invasion front. Oncogene. 2001; 20(16):1981-9.
38. Wulfkuhle J D, Speer R, Pierobon M, Laird J, Espina V, Deng J, et al. Multiplexed cell signaling analysis of human breast cancer applications for personalized therapy. J. Proteome Res. 2008; 7:1508-1517.
39. Pierobon M, Calvert V, Belluco C, Garaci E, Deng J, Lise M, et al. Multiplexed Cell Signaling Analysis of Metastatic and Nonmetastatic Colorectal Cancer Reveals COX2-EGFR Signaling Activation as a Potential Prognostic Pathway Biomarker. Clin Colorectal Cancer. 2009; 8(2):110-7.
40. Silvestri A, Colombatti A, Calvert V S, Deng J, Mammano E, Belluco C, et al. Protein pathway biomarker analysis of human cancer reveals requirement for upfront cellular-enrichment processing. Lab Invest. 2010; 90(5):787-96.
41. McMillen E, Ye F, Li G, Wu Y, Yin G, Liu W. Epidermal growth factor receptor (EGFR) mutation and p-EGFR expression in resected non-small cell lung cancer. Exp Lung Res. 2010; 36(9):531-7.
42. Havaleshko D M, Smith S C, Cho H, Cheon S, Owens C R, Lee J K, et al. Comparison of global versus epidermal growth factor receptor pathway profiling for prediction of lapatinib sensitivity in bladder cancer. Neoplasia. 2009; 11(11):1185-93.
43. Patnaik S K, Kannisto E, Knudsen S, Yendamuri S. Evaluation of MicroRNA expression profiles that may predict recurrence of localized stage I non-small cell lung cancer after surgical resection introduction. Cancer Res. 2010; 70(1):36-45.
44. Kadara H, Behrens C, Yuan P, Solis L M, Liu D, Gu X, et al. A five-gene and corresponding-protein signature for stage-I lung adenocarcinoma prognosis. Clin Cancer Res. 2010 Dec. 16.
45. Beer D G, Kardia S L, Huang C C, Giordano T J, Levin A M, Misek D E, et al. Gene-expression profiles predict survival of patients with lung adenocarcinoma. Nat Med. 2002; 8(8):816-24.
46. Dhillon T, Mauri F A, Bellezza G, Cagini L, Barbareschi M, North B V, et al. Overexpression of the mammalian target of rapamycin: a novel biomarker for poor survival in resected early stage non-small cell lung cancer. J Thorac Oncol. 2010; 5(3):314-9.
47. Yang D K, Son C H, Lee S K, Choi P J, Lee K E, Roh M S. Forkhead box M1 expression in pulmonary squamous cell carcinoma: correlation with clinicopathologic features and its prognostic significance. Hum Pathol. 2009; 40(4):464-70.
48. Tang J M, He Q Y, Guo R X Chang X J. Phosphorylated Akt overexpression and loss of PTEN expression in non-small cell lung cancer confers poor prognosis. Lung Cancer. 2006 February; 51(2):18×1-91.
49. Yoshizawa A, Fukuoka J, Shimizu S, Shilo K, Franks T J, Hewitt S M, et al Overexpression of phospho-eIF4E is associated with survival through AKT pathway in non-small cell lung cancer. Clin Cancer Res. 2010; 16(1):240-8.

All publications, including issued patents and published patent applications, and all database entries identified by url addresses or accession numbers are incorporated herein by reference in their entireties.

Although this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method of identifying a non-small cell lung carcinoma (NSCLC) patient with an aggressive node-negative (N0) tumor and a likelihood of a poor overall-survival comprising the step of:
determining if one or more of the proteins listed in Table 4 are activated in tumor cells obtained from the patient's tumor, wherein the activation of one or more of the proteins indicates that the patient has an aggressive N0 tumor and is likely to have a poor overall-survival.

2. The method of claim 1 wherein the activation of the one or more proteins is determined by measuring the phosphorylation, total amount, or cleavage of the proteins.

3. The method of claim 2 wherein the activation of the one or more proteins is compared to a series of calibrated standards wherein the standards contain predetermined amounts of the one or more proteins such that the value obtained for each patient sample is interpolated to the calibrator in order to generate a calibrated value.

4. The method of claim 3 wherein the calibrated value is compared to population data or reference standards with known low and high amounts of the target protein such that a determination of high and low levels of the given protein in Table 4 can be made.

5-13. (canceled)

14. The method of claim 1 wherein the tumor is an adenocarcinoma.

15. The method of claim 1 wherein the activation of two or more of the proteins listed in Table 4 is determined.

16-19. (canceled)

20. The method of claim 1 wherein the one or more proteins are selected from the proteins listed in Table 6 and their activation is determined by measuring their phosphorylation.

21. The method of claim 20 wherein the phosphorylation of two or more of the proteins listed in Table 6 is measured.

22-25. (canceled)

26. The method of claim 20 wherein at least one protein is selected from each of at least two of the separate signaling groups shown in Table 6.

27-30. (canceled)

31. The method of claim 20 wherein all of the proteins are from one of the signaling groups shown in Table 6.

32. A method for selecting a treatment for an NSCLC patient with an N0 tumor comprising the step of:
determining if one or more of the proteins listed in Table 4 are activated in tumor cells obtained from the patient's tumor, wherein the activation of one or more of the proteins indicates that the patient should be treated with aggressive or targeted therapy.

33. The method of claim 32 wherein the therapy comprises the administration to the patient of a therapeutically effective amount of a therapeutic agent in addition to surgery.

34-37. (canceled)

38. A method for treating an NSCLC patient with an N0 tumor comprising the steps of:
determining if one or more of the proteins listed in Table 4 are activated in tumor cells obtained from the patient's tumor, wherein the activation of one or more of the proteins indicates that the patient has an aggressive N0 tumor and is likely to have a poor overall-survival; and
treating the patient with aggressive or targeted therapy, if the patient has an aggressive N0 tumor and is likely to have a poor overall-survival.

39. (canceled)

40. The method of claim 39 wherein the therapy comprises the administration to the patient of a therapeutically effective amount of one or more chemotherapeutic agents that target one or more of the proteins listed in Table 4.

41. The method of claim 40 wherein the chemotherapeutic agent is selected from the agents listed in Table 7 or Table 8.

42.-45. (canceled)

46. A kit for selecting a treatment for an NSCLC patient with a node-negative tumor comprising: (i) one or more reagents for determining the activation of one or more of the proteins listed in Table 4 in tumor cells obtained from the patient, and (ii) instructions for performing the assay.

47. The kit of claim 46 wherein the reagents are antibodies.

48. The kit of claim 46 wherein the reagents are for measuring the phosphorylation level of one or more of the proteins listed in Table 6.

49. A pharmaceutical composition for treating an NSCLC patient with an aggressive N0 tumor and a likelihood of a poor overall-survival comprising a therapeutically effective amount of: (i) a chemotherapeutic agent that targets one or more of the proteins listed in Table 4, and (ii) a pharmaceutically acceptable carrier.

50. The pharmaceutical composition of claim 49 wherein the chemotherapeutic agent is selected from the agents listed in Table 7 or Table 8.

51. The pharmaceutical composition of claim 49 wherein the chemotherapeutic agent targets one or more of the proteins listed in Table 6.

52. The pharmaceutical composition of claim 51 wherein the chemotherapeutic agent is selected from the agents listed in Table 7 or Table 8.

53. A method of using a kit to select a treatment for an NSCLC patient with a node-negative tumor, wherein the kit comprises: (i) one or more reagents for determining the activation of one or more of the proteins listed in Table 4 in tumor cells obtained from the patient, and (ii) instructions for performing the assay, comprising the step of:
contacting the reagents in the kit with proteins obtained from tumor cells obtained from the patient,
determining if one or more of the proteins listed in Table 4 are activated in the tumor cells, and
if one or more of the proteins are activated, selecting an aggressive or targeted therapy for the patient.

54. The method of claim 53 wherein the activation of the one or more proteins is determined by measuring the phosphorylation, total amount, or cleavage of the proteins.

55. The method of claim 54 wherein the activation of the one or more proteins is compared to a series of calibrated standards wherein the standards contain predetermined amounts of the one or more proteins such that the value obtained for each patient sample is interpolated to the calibrator in order to generate a calibrated value.

56. The method of claim 53 wherein the tumor is an adenocarcinoma.
